(19) United States
(12) Patent Application Publication
(10) Pub. No.: US 2023/0248949 A1
(43) Pub. Date: Aug. 10, 2023

(54) TREATMENT METHOD AND TREATMENT SYSTEM

(71) Applicant: OLYMPUS MEDICAL SYSTEMS CORP., Tokyo (JP)

(72) Inventors: Masato NARISAWA, Hino-shi (JP); Ryosuke ITO, Kodaira-shi (JP); Yasushi YAMASAKI, Okayama-shi (JP)

(73) Assignee: OLYMPUS MEDICAL SYSTEMS CORP., Tokyo (JP)

(21) Appl. No.: 18/105,126

(22) Filed: Feb. 2, 2023

Related U.S. Application Data
(60) Provisional application No. 63/307,221, filed on Feb. 7, 2022.

(51) Int. Cl. A61M 25/10; A61M 1/00

(52) U.S. Cl. CPC A61M 25/1011; A61M 1/772; A61M 2025/105; A61M 2025/1052; A61M 2205/3337; A61M 2210/1064

(57) ABSTRACT

A treatment method includes: inserting a first closure and a second closure into a tubular organ, wherein the tubular organ includes at least one affected site; contacting an inner wall of the tubular organ with the first closure and the second closure to close the tubular organ with the at least one affected site located between the first closure and the second closure; with the tubular organ closed by the first closure and the second closure, aspirating a gas located in the tubular organ between the first closure and the second closure; and with the tubular organ closed by the first closure and the second closure, delivering a liquid medicine into the tubular organ between the first closure and the second closure.

### CROSS-REFERENCE TO RELATED APPLICATIONS

The present application claims priority based on U.S. Pat. Provisional Application No. 63/307,221 filed in the United States on Feb. 7, 2022, the entire contents of which are incorporated herein by reference.

### BACKGROUND

### Technical Field

The present disclosure relates to a treatment method and a treatment system. The present disclosure is suitable for simultaneously treating multiple affected sites in a tubular organ.

### Background Art

Colonic diverticulum is in principle a benign disease, but it may bleed due to inflammation or local stress, and if bleeding cannot be stopped by conservative treatment such as fasting and intestinal rest, endoscopic hemostasis (clip hemostasis, etc.) is necessary.

Large numbers of colonic diverticula often occur. If bleeding occurs in such a case, it may be difficult to specify the bleeding point by endoscopic observation or the like.

There is a technology for delivering medicine to a certain range within a tubular organ. Japanese Unexamined Patent Application, First Publication No. H5-42224 discloses a device with two balloons. The two balloons are inflated within the blood vessel, and medicine such as heparin can be administered into the blood-blocked space through an opening provided between the balloons.

### SUMMARY

A first aspect of the present disclosure is a treatment method.

This treatment method includes: inserting a first closure and a second closure into a tubular organ, wherein the tubular organ includes at least one affected site; contacting an inner wall of the tubular organ with the first closure and the second closure to close the tubular organ with the at least one affected site located between the first closure and the second closure; with the tubular organ closed by the first closure and the second closure, aspirating a gas located in the tubular organ between the first closure and the second closure; and with the tubular organ closed by the first closure and the second closure, delivering a liquid medicine into the tubular organ between the first closure and the second closure.

Another treatment method includes: inserting a tube including a first closure and a second closure into a tubular organ including at least one diverticulum; in a state in which the diverticulum is located between the first closure and the second closure, contacting an inner wall of the tubular organ with the first closure and the second closure to form a closed space between the first closure and the second closure; aspirating a gas in the diverticulum and the closed space to place the closed space under a negative pressure; and delivering a liquid medicine into the closed space to suppress bleeding in the diverticulum.

Another treatment system includes: inserting a catheter including a first closure and an overtube including a second closure into a tubular organ including at least one affected site; contacting an inner wall of the tubular organ with the first closure and the second closure to close the tubular organ with the at least one affected site located between the first closure and the second closure; with the tubular organ closed by the first closure and the second closure, aspirating a gas located in the tubular organ between the first closure and the second closure; and with the tubular organ closed by the first closure and the second closure, delivering a liquid medicine into the tubular organ between the first closure and the second closure.

### BRIEF DESCRIPTION OF THE DRAWINGS

FIG. 1 is a schematic diagram showing a device used in a treatment method according to one embodiment of the present disclosure.

FIG. 2 is a cross-sectional view taken along line I-I of FIG. 1.

FIG. 3 is a cross-sectional view taken along line II-II of FIG. 1.

FIG. 4 is a diagram showing an example of a control system of the device.

FIG. 5 is a flow chart showing a main flow of the treatment method.

FIG. 6 is a schematic diagram showing Step B of the treatment method.

FIG. 7 is a schematic diagram showing Step C of the treatment method.

FIG. 8 is a schematic diagram showing a process of the treatment method.

FIG. 9 is a schematic diagram showing Step D of the treatment method.

FIG. 10 is a graph showing an example of changes in values of a pressure gauge provided on the device in the treatment method.

FIG. 11 is a schematic diagram showing a modification of the device.

FIG. 12 is a cross-sectional view taken along line III-III of FIG. 11.

FIG. 13 is a cross-sectional view taken along line IV-IV of FIG. 11.

FIG. 14 is a diagram showing an example of a device control system according to a modification.

FIG. 15 is a schematic diagram showing the device in a state where a balloon is inflated.

FIG. 16 is a schematic diagram showing an overtube and a catheter used in a treatment method according to a second embodiment of the present disclosure.

FIG. 17 is a cross-sectional view taken along line III-III of FIG. 16.

FIG. 18 is a diagram showing an airtight valve.

FIG. 19 is a flow chart showing the main flow of the treatment method.

FIG. 20 is a schematic diagram showing step A-1 of the treatment method.

FIG. 21 is a schematic diagram showing step B-1 of the treatment method.

FIG. 22 is a schematic diagram showing step A-2 of the treatment method.

FIG. 23 is a schematic diagram showing step B-2 of the treatment method.

FIG. 24 is a schematic diagram showing step C of the treatment method.

FIG. 25 is a schematic diagram showing step X of the treatment method.

FIG. 26 is a schematic diagram showing step D of the treatment method.

FIG. 27 is a diagram showing another insertion mode of the catheter.

FIG. 28 is a diagram showing another insertion mode of the catheter.

FIG. 29 is a schematic diagram showing step C using an aspiration catheter.

FIG. 30 is a diagram showing a balloon cover.

FIG. 31 is a schematic diagram of a device used in a treatment method according to a third embodiment.

FIG. 32 is a schematic diagram of the device in which a pressure monitor is operated.

FIG. 33 is a diagram showing a modification of the pressure monitor.

### DETAILED DESCRIPTION

### First Embodiment

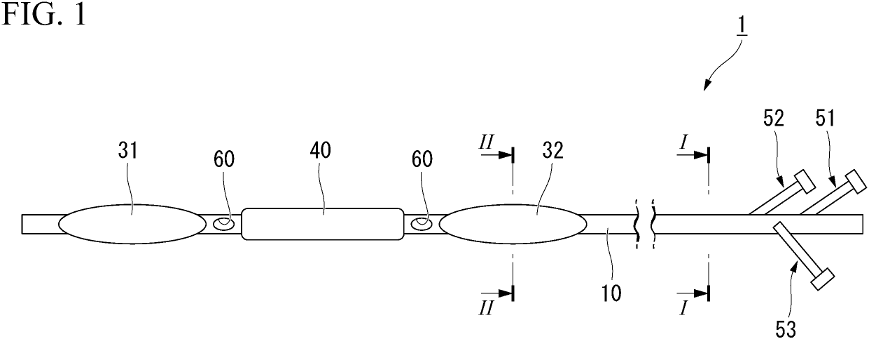

A first embodiment of the present disclosure will be described with reference to FIGS. 1 to 12.

FIG. 1 is a schematic diagram of a device 1 used in the treatment method according to this embodiment. The treatment method according to this embodiment is not limited to using the device 1, but can be performed by using the device 1.

The device 1 includes a long tube 10 and three balloons attached to the tube 10.

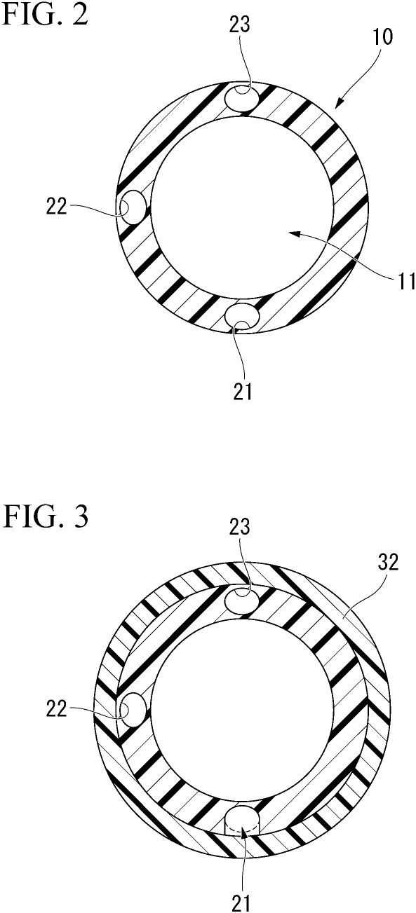

FIG. 2 is a cross-sectional view taken along line I-I in FIG. 1. The tube 10 is a multi-lumen tube having four lumens, a main lumen 11 having the largest inner diameter and three sub-lumens (lumens) formed in the wall, as shown in FIG. 2. Details of the sub-lumen will be described later.

Of the three balloons, a first balloon (first closing member, first closure) 31 closest to the distal end and a second balloon (second closing member, second closure) 32 furthest from the distal end are made of easily extensible material such as silicone or elastomer. A third balloon (auxiliary balloon) 40 located between the first balloon 31 and the second balloon may be made of the same material as the first balloon 31, but is made of a material such as vinyl chloride that is difficult to expand.

FIG. 3 is a cross-sectional view taken along line II-II of FIG. 1. Of the three sub-lumens, a first lumen (inflation lumen) 21 opens on the outer peripheral surface of the tube 10 at the site where the first balloon 31 and the second balloon 32 are attached, and communicates with the first balloon 31 and the second balloon 32. Although FIG. 3 shows a portion where the first lumen 21 and the second balloon 32 communicate, the first lumen 21 and the first balloon 31 communicate in a similar manner. The first lumen 21 extends to a first port 51 provided on the proximal side of the device 1, and can inflate the first balloon 31 and the second balloon 32 when fluid is supplied to the first port 51.

A second lumen (second inflation lumen) 22 opens on the outer peripheral surface of the site where the third balloon 40 is attached and communicates with the third balloon 40. The second lumen 22 extends to a second port 52 provided on the proximal side of the device 1, and can inflate the third balloon 40 when fluid is supplied to the second port 52.

A third lumen (aspiration lumen, liquid delivery lumen) 23 communicates with an opening 60 provided on the outer peripheral surface of the tube between the first balloon 31 and the third balloon 40 and between the third balloon 40 and the second balloon 32. The third lumen 23 extends to a third port 53 provided on the proximal side of device 1.

The first port 51, the second port 52, and the third port 53 are connected to the control system.

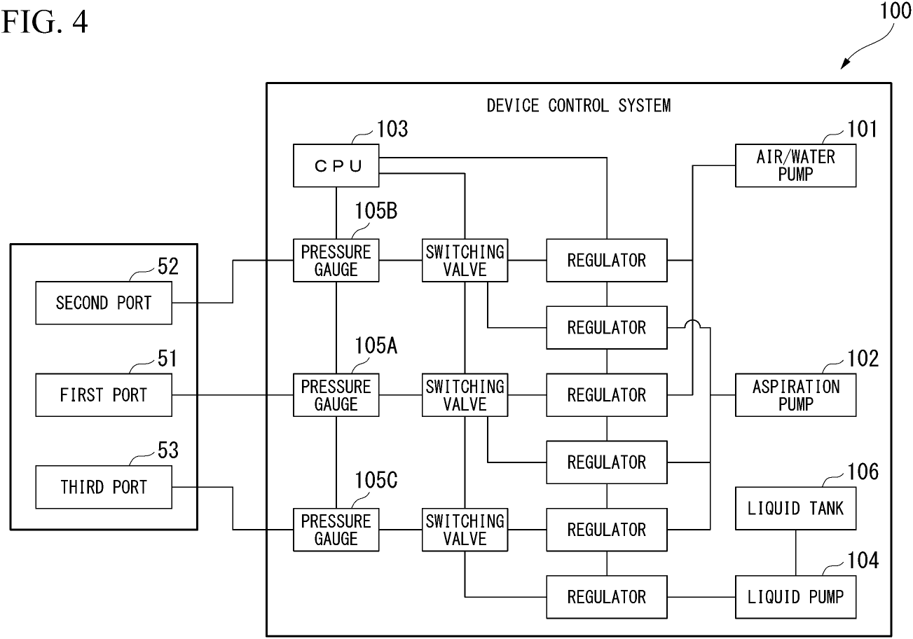

An example of the control system is shown in FIG. 4. In one example, the control system includes pressure gauges, switching valves, regulators, various pumps, and a CPU that controls them. The CPU controls all of the switching valves, regulators and pumps shown in FIG. 4.

In a control system 100 shown in FIG. 4, an air/water pump 101 and an aspiration pump 102 are connected to the first port 51 and the second port 52. The flow and direction of the fluid are controlled by a CPU 103 operating switching valves installed on paths leading to each port. Pressure gauges 105A, 105B, and 105C are installed in the conduits associated with the respective ports, and the internal pressures of the sub-lumens 21, 22, and 23 are obtained. The CPU 103 controls each regulator according to the measured value of each pressure gauge to control the internal pressure of the target sub-lumen to a desired value.

A liquid pump 104 and the aspiration pump 102 are connected to the third port 53. The CPU 103 can control liquid delivery and aspiration from the opening 60 by controlling each regulator according to the measured value of the pressure gauge.

The number and arrangement of switching valves and regulators in the control system 100 are not limited to the mode shown in FIG. 4, and can be set as appropriate.

If the material and thickness of the first balloon 31 and the third balloon 40 are the same, the regulator connected to the air/water pump 101 can be omitted.

A liquid tank 106 is connected to the liquid pump 104. The liquid tank 106 contains medicine that can be used for treatment. In this embodiment, the following agents that exert a hemostatic effect can be used.
Bioabsorbable topical hemostatic agents, including gelatin and cross-linked gelatin
Bioadhesives containing gelatin, crosslinked gelatin, etc.
Bioadhesive that contains fibrinogen and thrombin and gels upon reaction
Injectable gel that gels inside the body. There are those that gel by reaction between collagen and a cross-linking agent, those that gel by reacting with moisture in the living body, those that gel by temperature change, and the like, and any of them can be used.
Coating materials such as hydrogels containing urethane-based polymers and carboxymethyl cellulose (CMC)

The above is just an example, and in addition to these, a liquid or viscous liquid biocompatible medicine that exhibit local hemostatic effect, bioadhesiveness, wound sealing effect, wound healing effect, etc., can be delivered via the auxiliary lumen, and thus can be used for the treatment method according to the present embodiment. In the case of medicine that causes gelation or adhesiveness due to the reaction of multiple substances, a configuration in which the control system includes a plurality of liquid tanks and is selectively connected to the liquid pump 104 by the switching valve or the like, a configuration in which a plurality of liquid tanks is connected to different sub-lumens, or the like can be adopted.

The device 1 and the control system 100 constitute a treatment system according to this embodiment.

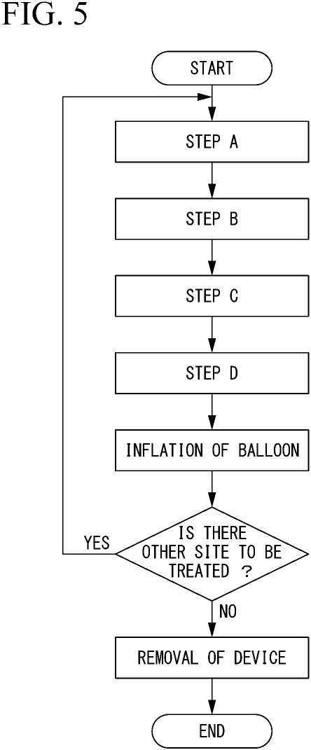

FIG. 5 is a flow chart showing the main flow of the treatment method according to this embodiment.

The treatment method of the present embodiment using the device 1 will be described using an example of collective treatment of a plurality of diverticula occurring in the large intestine. A diverticulum is a concave lesion formed on the inner surface of the large intestine.

First, the endoscope is passed through the main lumen 11 of the tube 10. At this point, the device 1 and control system may not be connected.

The operator inserts the endoscope into the large intestine to be treated and advances the distal end of the endoscope to the site to be treated.

The operator then inserts the tube 10 into the large intestine (Step A). Further, the tube 10 is advanced along the endoscope so that the portion of the tube 10 provided with the balloon reaches the site to be treated.

Advancement of the endoscope and advancement of the tube 10 may be performed in parallel.

Next, the operator connects the device 1 to the control system and activates the control system. When connecting, as shown in FIG. 4, the first port 51 and the second port 52 are connected to the pipeline to which the air/water pump 101 is connected, and the third port 53 is connected to the pipeline to which the liquid pump 104 is connected. The connection between the device 1 and the control system can be made at an appropriate timing beforehand.

As an initialization operation, the control system 100 according to the present embodiment activates the liquid pump 104 and delivers the liquid to the switching valve. As a result, the gas in the pipeline from the liquid pump 104 to the switching valve is removed.

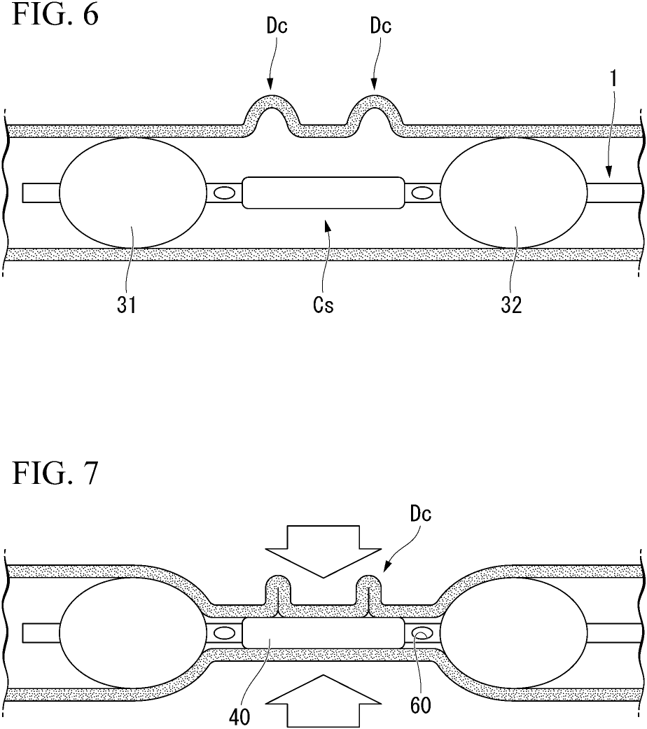

Next, the operator supplies gas or liquid to the first balloon 31 and the second balloon 32 and makes them inflated (Step B). The inflated first balloon 31 and second balloon 32 contact the inner wall of the large intestine to close the lumen of the large intestine, as shown in FIG. 6. As a result, a closed space Cs is formed between the first balloon 31 and the second balloon 32, as shown in FIG. 6.

As will be described later, the device 1 can collectively treat the affected site located within the closed space Cs. Therefore, in Step A, the position of the tube 10 is determined so that at least one diverticulum Dc is located between the first balloon 31 and the second balloon 32.

The expansion and stopping of the first balloon 31 and the second balloon 32 in Step B may be manually performed by the operator, but automatic control by the control system 100 is also possible. The inflated first balloon 31 and second balloon 32 become difficult to be inflated after coming into contact with the inner wall of the large intestine, and the internal pressure rises quickly. Therefore, by detecting this change point from the value of the pressure gauge connected to the first port 51 and operating the air/water pump 101 by the CPU 103, the fluid supply can be stopped at the point of contact with the inner wall of the large intestine and the inflated state can be maintained.

Next, the operator makes the gas in the closed space Cs aspirated through the opening 60 (Step C). The control system 100 operates the switching valve and regulator of the line connected to the third port 53 to perform aspiration by the aspiration pump 102. By Step C, the internal pressure of the closed space Cs is reduced to a negative pressure. As a result, as shown in FIG. 7, the large intestine in the closed space is deflated, the inner wall approaches the third balloon 40, and the outer shape is reduced. Furthermore, gas in the diverticulum Dc is also aspirated.

In Step C, gas is aspirated, but if necessary, not only gas but also unnecessary body fluids in the digestive tract may be aspirated.

The aspiration from the opening 60 may be manually stopped by the operator, but automatic control by the control system 100 is also possible. The numerical value of the pressure gauge of the pipeline connected to the third port 53 decreases as the gas is aspirated. When the gas in the closed space Cs is almost gone, the numerical value of the pressure gauge 105C drops rapidly. When the gas in the closed space Cs is almost gone, the inner wall of the large intestine comes into contact with the opening 60 and closes the opening 60. Therefore, by detecting such a change and causing the CPU 103 to operate the aspiration pump 102, the aspiration of the gas can be stopped while the pressure inside the closed space Cs is sufficiently reduced. In such a case, the CPU 103 functions as a control device having an aspiration mode in which the aspiration pump is operated to aspirate gas from the sub-lumen while the liquid delivering operation of the liquid pump is stopped and a liquid delivering mode in which the liquid pump is operated to deliver the liquid to the sub-lumen while the aspiration operation of the aspiration pump is stopped.

When the operator manually performs control, for example, the operator may turn on/off the aspiration pump 102 based on the amount of gas aspirated from the aspiration pump 102 or the like.

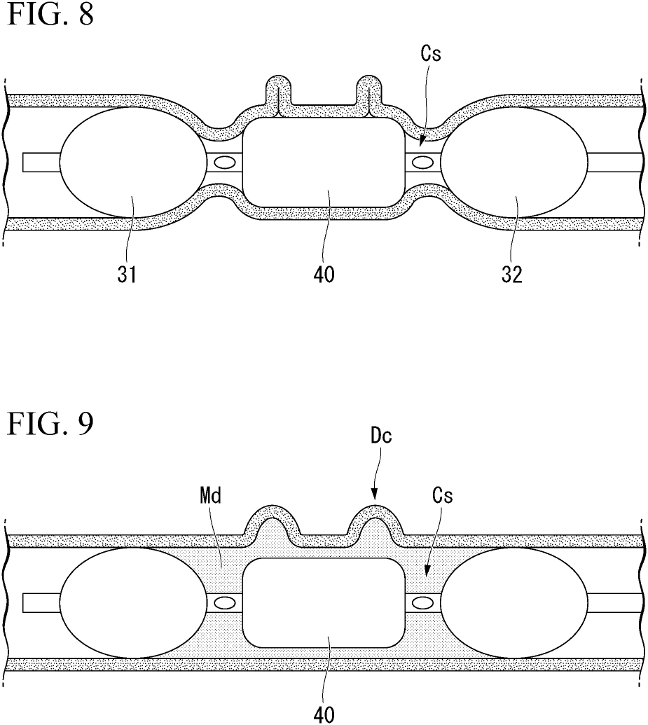

Next, the operator makes the third balloon 40 inflated while maintaining the inflated states of the first balloon 31 and the second balloon 32, as shown in FIG. 8. The control system 100 operates the switching valve to connect the air/water pump 101 and the second port 52, and delivers the fluid to the third balloon 40 by the air/water pump 101.

When the third balloon is inflated, the outer diameter of the large intestine in the closed space increases and approaches the value before Step C, but since gas is not introduced into the closed space Cs, a state of low internal pressure is maintained.

Further, the operator operates the liquid pump 104 to deliver the medicine into the closed space Cs through the opening 60 (Step D). By the delivery of the medicine, the internal pressure gradually increases in the closed space Cs. When the internal pressure increases to some extent, the medicine also enters between the third balloon 40 and the inner wall of the large intestine. At this time, since the gas in the diverticulum Dc has already been aspirated, the medicine Md smoothly enters the diverticulum Dc as well, as shown in FIG. 9. As a result, even when a plurality of diverticula Dc are present in the closed space Cs, the medicine Md can be collectively administered without considering the position of each diverticulum Dc.

By Step D, the diverticulum Dc is in a state where bleeding is stopped or bleeding is difficult. As a result, bleeding of the diverticula Dc is suppressed as a whole.

The delivery of the medicine may be manually stopped by the operator, but automatic control by the control system 100 is also possible. The numerical value of the pressure gauge of the pipeline connected to the third port 53 rises as the medicine is delivered. When the numerical value exceeds the numerical value of the pressure gauge 105A of the first port 51, that is, the internal pressure of the first balloon 31 and the second balloon 32, since the medicine leaks out of the closed space Cs from between the first balloon 31 and the second balloon 32 and the inner wall of the large intestine, the value of the pressure gauge 105C then repeats up and down near the value of the pressure gauge 105A. Therefore, by detecting such a change and causing the CPU 103 to operate the liquid pump 104, delivery of the medicine can be stopped in a state where the closed space Cs is sufficiently filled with the medicine.

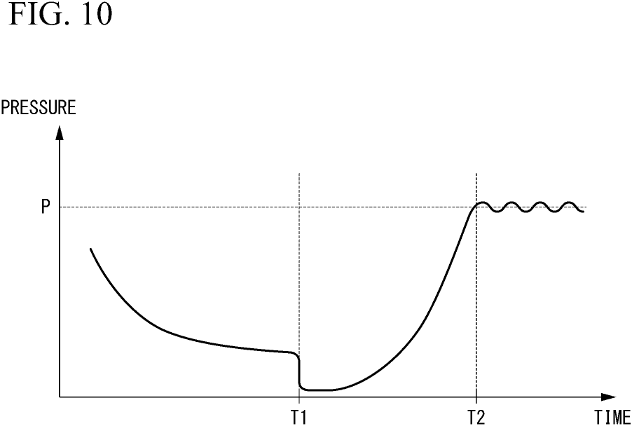

FIG. 10 shows an example of the variation pattern of the numerical value of the pressure gauge 105C. The timing T1 at which the numerical value rapidly decreases can be used as a criterion for stopping aspiration, and the timing T2 at which the numerical value repeats up and down around a predetermined value P can be used as a criterion for stopping medicine delivery.

After stopping the delivery of the medicine, the closed space Cs is sufficiently filled with the medicine for a predetermined time (for example, several seconds to several minutes) until the medicine exerts its hemostatic effect. Thereafter, the balloons 31, 32, 40 are deflated and the device 1 is removed from the body, and the treatment method according to this embodiment is completed. If there are other sites to be treated, the device 1 may be moved to the next site to be treated without being removed from the body.

If there is no problem in flowing the used medicine into the large intestine, the medicine remaining in the closed space Cs may not be recovered. However, if it is desired to prevent the medicine from coming into contact with other sites in the large intestine, the medicine can be collected by aspiration from the opening 60 before deflating the first balloon 31 and the second balloon 32.

As described above, in the treatment method according to the present embodiment, a plurality of affected sites present on the inner wall of the closed space Cs can be treated at once by supplying the medicine at once. Therefore, the operator can perform treatment without specifying which of the plurality of affected sites is bleeding. Further, by providing a blanket treatment, in addition to hemostasis of bleeding diverticula, diverticula that are not currently bleeding but are likely to bleed can be simultaneously treated prophylactically.

In addition, in Step C, since the gas in the closed space is aspirated to reduce the internal pressure, the gas present in the concave affected site such as the diverticulum is also aspirated and removed. As a result, in the following Step D, the medicine can be supplied to the inside of all affected sites regardless of the position of the affected site. That is, even if the patient’s body position during treatment is a concave affected site located vertically upward, by performing Step D after Step C, the medicine can be suitably supplied without changing the patient’s body position.

Therefore, the treatment method of this embodiment eliminates the complexity of the patient and the operator, shortens the required time, and improves the treatment effect.

The device used in the treatment method of this embodiment is not limited to the device 1 described above.

FIG. 11 shows a device 201 of a modified example. In the following description, the same reference numerals are given to the same configurations as those already described, and redundant descriptions will be omitted.

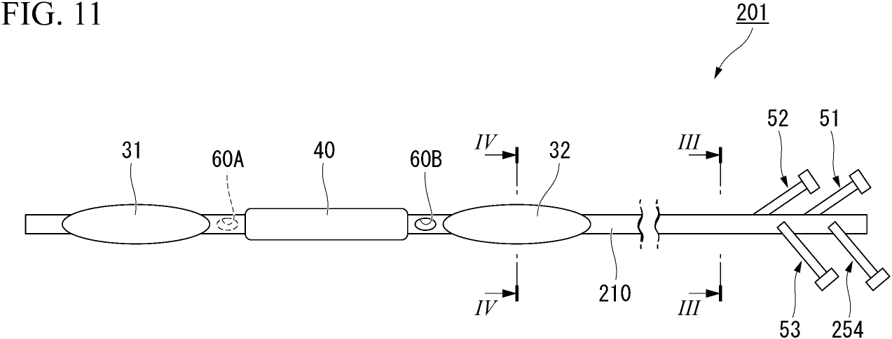

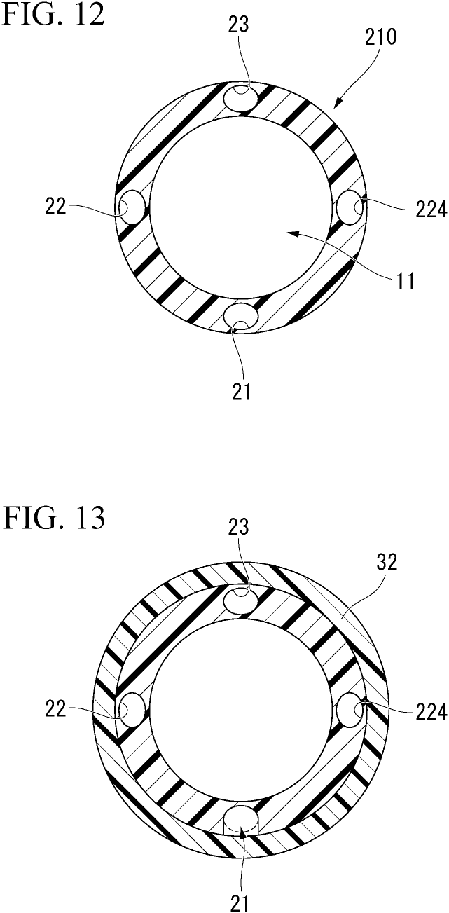

FIG. 12 is a cross-sectional view taken along line III-III of FIG. 11. FIG. 13 is a cross-sectional view taken along line IV-IV of FIG. 11. The tube 210 of the device 201 has four sub-lumens. The third lumen 23 communicates only with the opening 60A on the distal side, and the opening 60B on the proximal side communicates with the fourth lumen 224. The fourth lumen 224 extends to a fourth port 254 provided on the proximal side of the device 201.

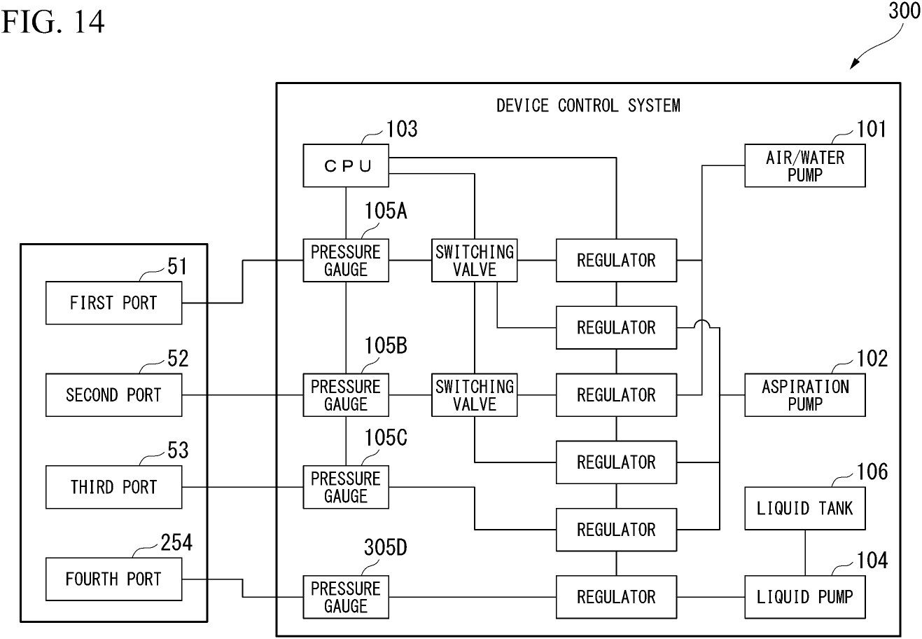

An example of the control system of the device 201 is shown in FIG. 14. In a control system 300, a pressure gauge 305D is provided in a conduit leading to the fourth port 254, and the liquid pump 104 is connected. Furthermore, the switching valve of the pipeline connected to the third port 53 is omitted. That is, in the device 201, the liquid delivery lumen and the aspiration lumen are provided independently.

In the treatment method using the device 201, Step C is performed using the opening 60A, and Step D is performed using the opening 60B. Therefore, when performing the treatment method according to this embodiment using the device 201, Step C and Step D can be performed partially or completely in parallel.

The combination of the openings 60A and 60B and the sub-lumens connected may be reversed, and the configuration of the control system may be changed accordingly.

In the device used in this embodiment, the maximum diameter of the third balloon 40 when inflated can be set so as not to exceed the diameters of the first balloon 31 and the second balloon 32 in step B, as shown in FIG. 15. As a result, when the third balloon 40 is inflated, the third balloon 40 makes the inner wall of the large intestine separate from the first balloon 31 and the second balloon 32, which have been in close contact with the large intestine separate, and it is possible to suitably suppress the leakage of the medicine or the like caused by releasing the closed state of the closed space Cs.

Although the first embodiment of the present disclosure has been described above, the technical scope of the present disclosure is not limited to the above embodiments. Various changes can be added or deleted. In addition to the modifications described above, some further modifications are exemplified, but not exhaustive, and other modifications are possible. Two or more of these changes may be combined as appropriate, and may be combined with the changes described above.

In the treatment method according to the present disclosure, inflating the third balloon is not essential and may be omitted. Therefore, the device used may not have a third balloon. However, by inflating the third balloon, there is an advantage that the effective volume of the closed space can be reduced and the medicine can be supplied into the affected site with a smaller delivery amount of the medicine.

If no third balloon is used, at least two of Steps B, C and D may be performed partially in parallel or completely simultaneously.

The step of inflating the third balloon and Step D may be performed partially in parallel or completely simultaneously.

In the treatment method according to the present disclosure, it is not essential that the operation in each step be automatically performed by system control. For example, a mode in which a syringe or the like is connected to each port of the device and the operator manually performs each step is also included in the treatment method according to the present disclosure.

Each step of the treatment method according to the present disclosure can be performed without observing the affected site as long as the affected site is located between the first balloon and the second balloon. Therefore, there is no restriction on the positional relationship between the device and the endoscope during execution of the treatment method, and the endoscope may be removed from the device.

The target of the treatment method according to the present disclosure is not limited to the above-described colonic diverticulum, and can be applied to various affected sites formed in the digestive tract such as esophageal diverticulum. The treatment method according to the present disclosure is particularly effective when the affected site has a complicated shape and occurs in large numbers.
The closing member(closure) is not limited to the balloon described above. For example, an umbrella-shaped member or the like that can close a tubular organ by opening inside the tubular organ can be used.
The first balloon and the second balloon may communicate with different lumens. With such a configuration, the first and second balloons can be inflated and deflated independently.

The aspiration pump and the liquid pump may be connected to different lumens to separate the aspiration lumen and the liquid delivery lumen. In this case, the control device described above can independently control the operation of one of the aspiration pump and the liquid pump without considering the state of the other pump. A liquid delivering mode for starting the liquid delivering operation of the liquid pump may be provided as an operation mode.

Although the above embodiments show a device having a main lumen through which an endoscope is passed, even by devices such as balloon catheters that have a similar configuration and are used through the channels of an endoscope, the treatment method according to the present disclosure can be performed.

### Second Embodiment

A second embodiment of the present disclosure will be described with reference to FIGS. 16 to 26. In the following description, the same reference numerals are given to the same configurations as those already described, and redundant descriptions will be omitted.

FIG. 16 is a schematic diagram of the overtube 301 and a catheter 401 used in the treatment method according to this embodiment. The treatment method according to this embodiment is not limited to using the overtube 301 and the catheter 401, but can be suitably performed by using the overtube 301 and the catheter 401.

The overtube (balloon overtube) 301 includes a long tube 310 and a second balloon 332 attached to the tube 310.

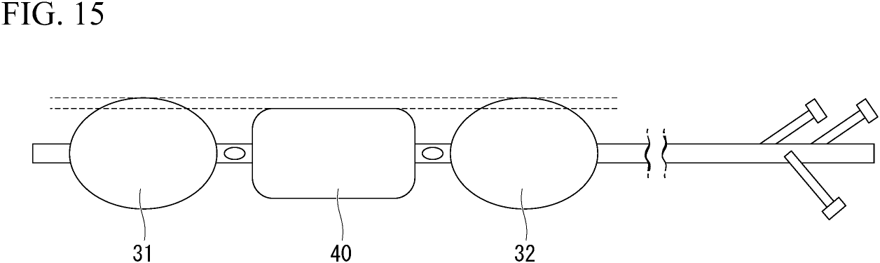

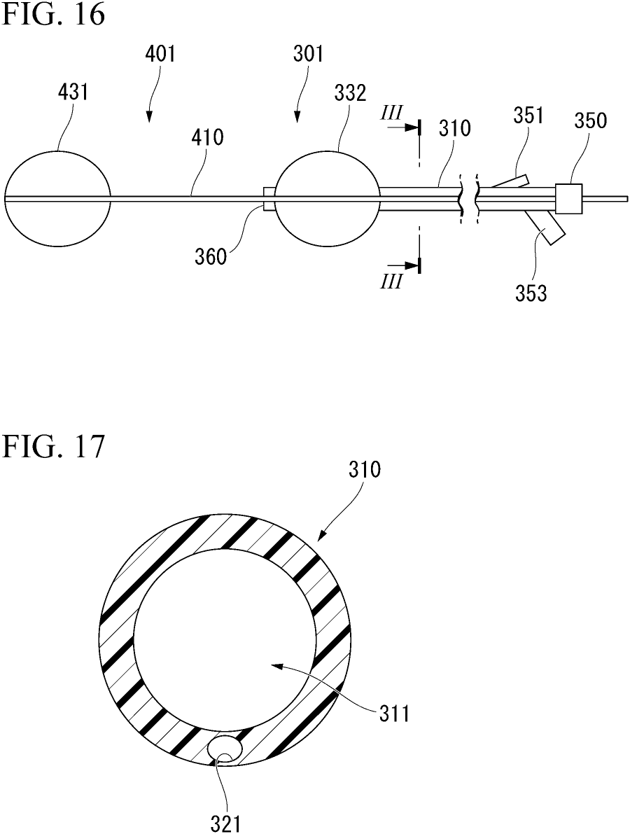

FIG. 17 is a cross-sectional view taken along line III-III in FIG. 16. The tube 310 is a multi-lumen tube having two lumens, a main lumen 311 having the largest inner diameter and one sub-lumen (lumen) formed in the wall, as shown in FIG. 2.

The second balloon (second closing member, second closure) 332 is provided at the distal end of the tube 310. The second balloon 332 is made of a stretchable material such as silicone or elastomer.

A first lumen (inflation lumen) 321, which is a sub-lumen, opens to the outer peripheral surface of the tube 310 where the second balloon 332 is attached and communicates with the second balloon 332. The first lumen 321 extends to a first port 351 provided on the proximal side of the overtube 301, and can inflate the second balloon 332 upon supplying fluid to the first port 351.

The main lumen 311 communicates with an opening 360 provided at the distal end of the tube. The main lumen 311 extends to a main port 350 and a third port 353 provided on the proximal side of the overtube 301.

FIG. 18 is a diagram showing an airtight valve 355.

The main port 350 is provided with the airtight valve 355. The diameter of the insertion passage of the airtight valve 355 is reduced when the catheter 401 is inserted, and expanded when the endoscope 500 is inserted. Note that the airtight valve 355 may be provided on the catheter 401 passing through the main port 350.

The first port 351 and third port 353 are connected to the same control system 100 as in the first embodiment. The air/water pump 101 and the aspiration pump 102 are connected to the first port 351. The liquid pump 104 and the aspiration pump 102 are connected to the third port 353. Note that the first port 351 and the third port 353 may be connected to a syringe S as shown in FIG. 18.

The catheter (balloon catheter) 401 includes a long tube 410 and a first balloon 431 attached to the tube 410. The catheter 401 can be passed through the main lumen 311 of the overtube 301 while the diameter of the first balloon 431 is reduced.

The tube 410 extends to a fifth port provided on the proximal side of the catheter 401, and the first balloon 431 can be inflated by supplying fluid to the fifth port. The port of tube 410 may be connected to control system 100 similar to the first embodiment.

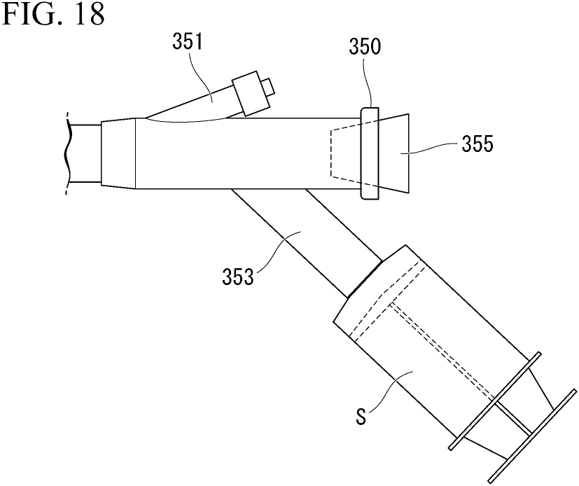

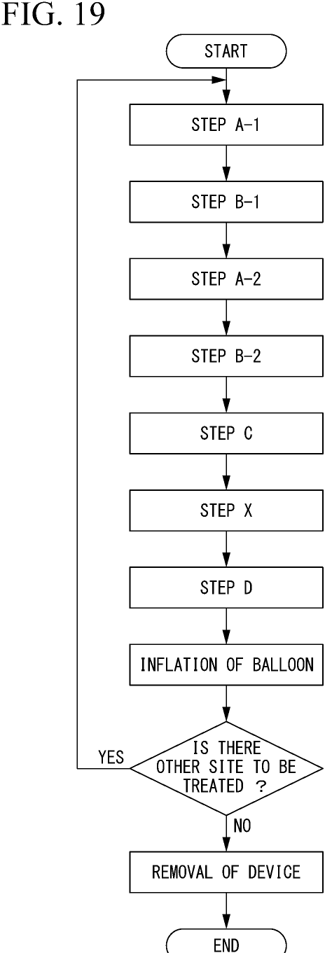

FIG. 19 shows a flow chart of the main flow of the treatment method according to this embodiment.

The treatment method of this embodiment using the overtube 301 and the catheter 401 will be described using an example of collectively treating a plurality of diverticula generated in the large intestine.

FIG. 20 is a schematic diagram showing step A-1 of the treatment method.

First, the endoscope 500 is passed through the main lumen 311 of the overtube 301. At this point, overtube 301 and control system 100 may not be connected.

The operator inserts the endoscope 500 into the large intestine to be treated, and advances the distal end of the endoscope 500 to the site to be treated.

Next, the operator inserts the tube 310 into the large intestine (step A-1). Further, the tube 310 is advanced along the endoscope 500 so that the portion of the tube 310 provided with the second balloon 332 reaches the site to be treated.

Advancement of the endoscope 500 and advancement of the tube 310 may be performed in parallel.

FIG. 21 is a schematic diagram showing step B-1 of the treatment method.

Next, the operator connects the overtube 301 to the control system 100 and activates the control system 100. When connecting, the first port 351 is connected to the pipeline to which the air/water pump 101 is connected, and the third port 353 is connected to the pipeline to which the liquid pump 104 is connected. The connection between the overtube 301 and the control system 100 can be made at any time before this.

Next, the operator supplies gas or liquid to the second balloon 332 to expand it (step B-1). The expanded second balloon 332 contacts the inner wall of the large intestine to close the lumen of the large intestine, as shown in FIG. 21.

As will be described later, the overtube 301 and the catheter 401 can treat the affected site located in front of the second balloon 332. Therefore, in step A-1, the position of the tube 310 is determined so that the diverticulum Dc to be treated is located in front of the second balloon 332.

The operator withdraws the endoscope 500 from the main lumen 311 after expanding the second balloon 332. Note that if the endoscope 500 does not interfere with the treatment in subsequent steps, the endoscope 500 does not have to be removed.

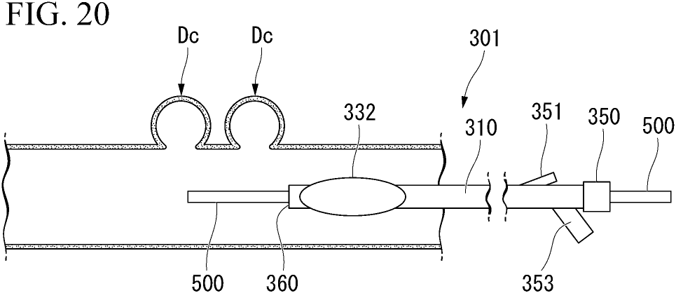

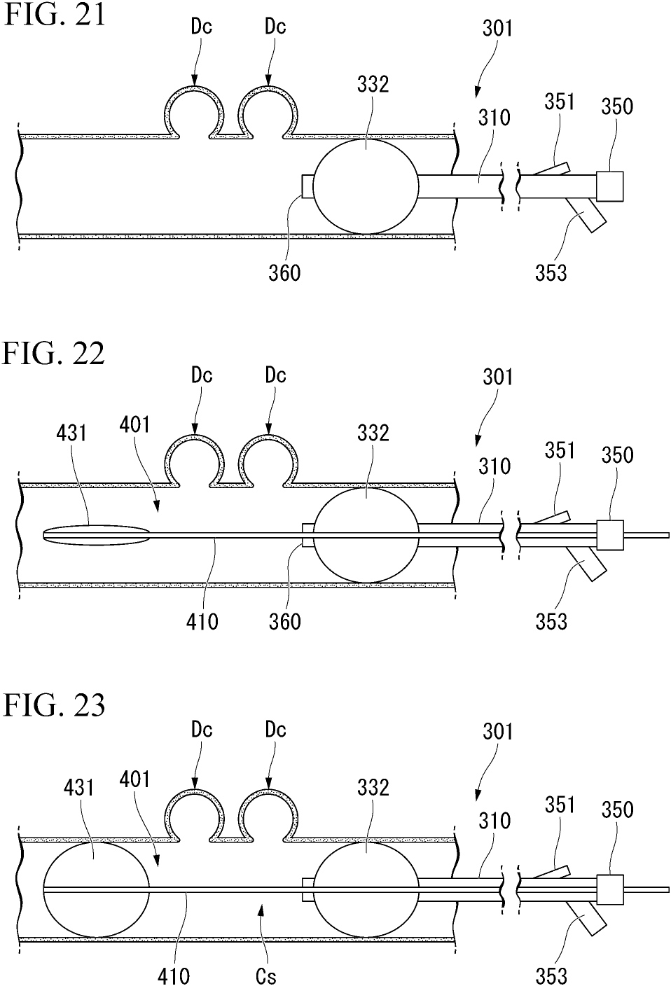

FIG. 22 is a schematic diagram showing step A-2 of the treatment method.

Next, the operator inserts the catheter 401 into the large intestine (step A-2). Specifically, the operator advances catheter 401 along main lumen 311 of overtube 301 to protrude from opening 360. The operator brings the site of the catheter 401 where the first balloon 431 is provided to the site to be treated.

The operator may expand the lumen by supplying gas from the third port 353 to the lumen before inserting the catheter 401 into the large intestine. By inserting the catheter 401 into the large intestine after expanding the lumen, it is possible to prevent the catheter 401 from contacting the lumen and damaging the lumen.

FIG. 23 is a schematic diagram showing step B-2 of the treatment method.

Next, the operator connects the fifth port of the tube 410 of the catheter 401 to the control system 100 and supplies gas or liquid to the first balloon 431 to expand it (step B-2). The expanded first balloon 431 contacts the inner wall of the large intestine to close the lumen of the large intestine. As a result, a closed space Cs is formed between the first balloon 431 and the second balloon 332.

As will be described later, the overtube 301 and the catheter 401 can treat the affected site behind the first balloon 431. Therefore, in step A-2, the position of the tube 410 is determined so that the diverticulum Dc to be treated is located behind the first balloon 431.

It is also possible to attach a memory to the proximal portion of the tube 410 of the catheter 401, and the operator can confirm the protrusion amount of the catheter 401 from the opening 360 of the overtube 301 by confirming the memory.

FIG. 24 is a schematic diagram showing step C of the treatment method.

Next, the operator aspirates the gas in the closed space Cs from the opening 360 (step C). The control system 100 operates the switching valve and the regulator of the line connected to the third port 353 to perform aspiration by the aspiration pump 102. By step C, the internal pressure of the closed space Cs is reduced to a negative pressure. As a result, as shown in FIG. 24, the large intestine in the closed space Cs contracts, the inner wall approaches the tube 410, and the outer shape shrinks. Furthermore, gas in the diverticulum Dc is also aspirated.

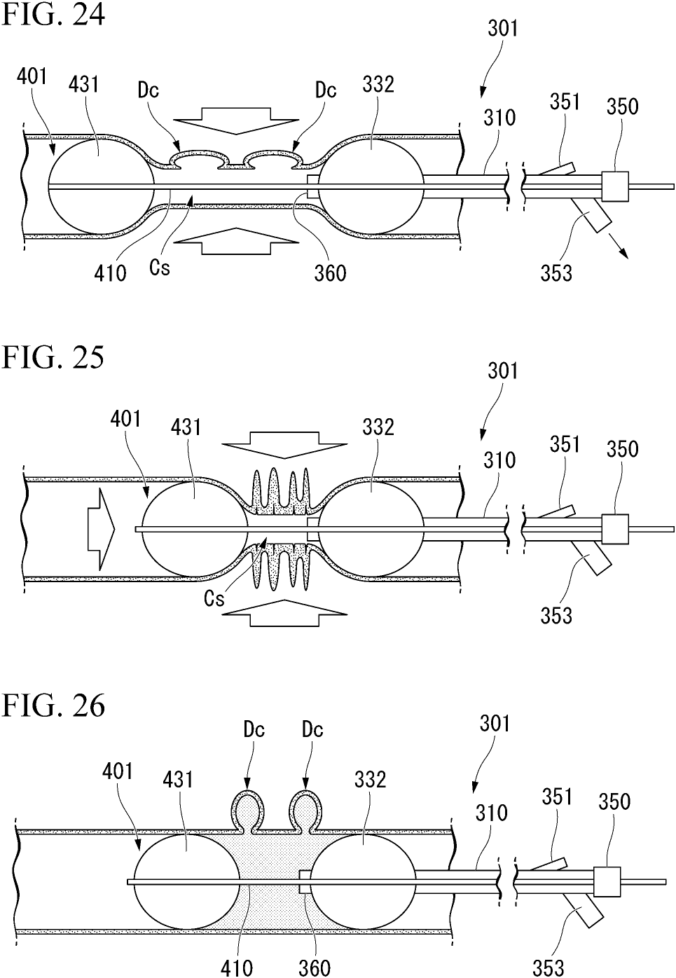

FIG. 25 is a schematic diagram showing step X of the treatment method.

Next, the operator pulls the catheter 401 toward the proximal side (proximal end side) to draw the intestinal tract toward the proximal side and shorten the closed space Cs (step X). By shortening the closed space Cs, the delivered amount of the medicine to be delivered in the next step D is reduced, and the treatment time is also reduced. Note that step X is an optional step, and is omitted if unnecessary.

Note that step X may be performed before step C. However, if step X is performed before step C, the inlet of the diverticulum Dc is blocked when the closed space Cs is shortened in step X, and the gas in the diverticulum Dc may not be sufficiently aspirated in step C. When performing step X after step C, the entrance of the diverticulum Dc is widened in step C, and the entrance of the diverticulum Dc in step X is less likely to be blocked. Therefore, it is desirable to perform step X after step C.

FIG. 26 is a schematic diagram showing step D of the treatment method.

Further, the operator operates the liquid pump 104 to deliver the medicine into the closed space Cs through the opening 360 (step D). The delivery of the medicine gradually increases the internal pressure in the closed space Cs. Since the gas in the diverticulum Dc has already been aspirated, the medicine Md smoothly enters the diverticulum Dc as well, as shown in FIG. 26. As a result, even when a plurality of diverticula Dc are present in the closed space Cs, the medicine Md can be collectively administered without considering the position of each diverticulum Dc.

By step D, the diverticulum Dc is in a state where bleeding is stopped or bleeding is difficult. As a result, bleeding of the diverticula Dc is suppressed as a whole.

The cross-sectional area of the main lumen 311 is larger than the cross-sectional area of the third lumen 23 to which the medicine is supplied in the first embodiment. Therefore, the treatment method of the second embodiment can efficiently supply the medicine to the closed space Cs even if the viscosity of the medicine is higher than that of the treatment method of the first embodiment. For the same reason, the treatment method of the second embodiment can more efficiently aspirate gas from the closed space Cs than the treatment method of the first embodiment.

As described above, in the treatment method according to the present embodiment, similarly to the treatment method of the first embodiment, a plurality of affected sites existing on the inner wall of the closed space Cs can be treated collectively by supplying the medicine at once. In the treatment method according to this embodiment, by changing the relative positions of the overtube 301 and the catheter 401, it is possible to adjust the position of the first balloon 431 with respect to the second balloon 332, and the closed space Cs formed between the first balloon 431 and the second balloon 332 can be adjusted according to the diverticulum Dc to be treated. Furthermore, by shortening the closed space Cs in step X, the delivery amount of the medicine to be delivered is reduced, and the treatment time is also reduced. As a result, the medicine can be supplied to the diverticulum Dc to be treated.

Therefore, the treatment method of this embodiment eliminates the complexity of the patient and the operator, shortens the required time, and improves the treatment effect.

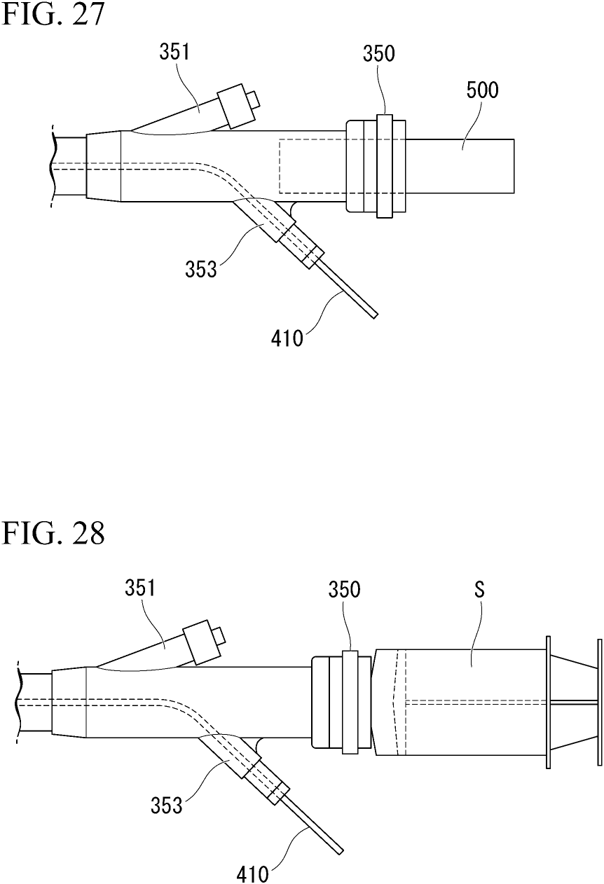

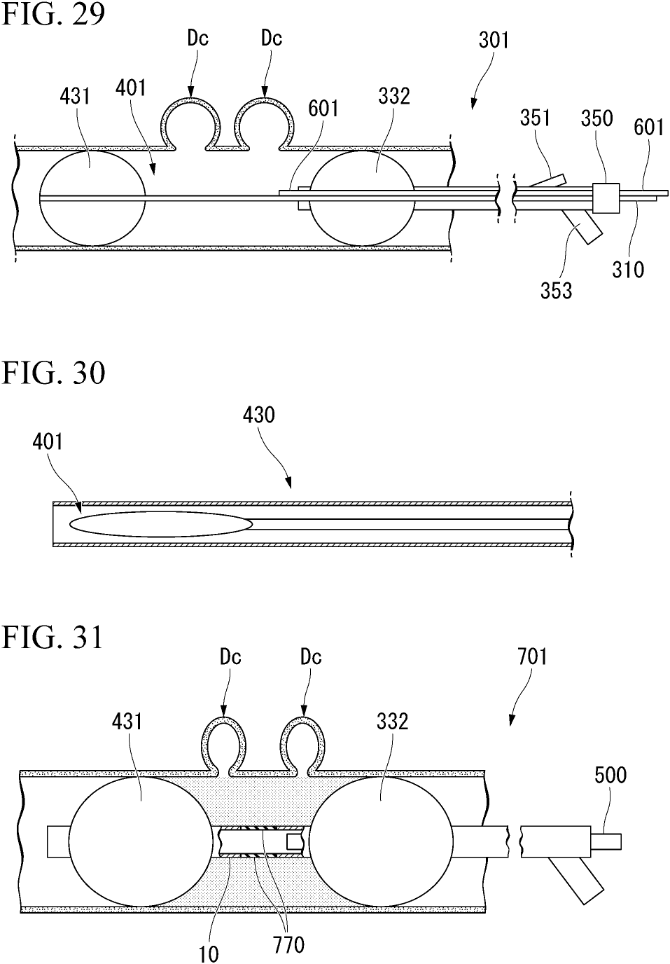

As described above, the second embodiment of the present disclosure has been described, but the technical scope of the present disclosure is not limited to the above-described embodiment, and the combination of the constituent elements can be changed without departing from the scope of the present disclosure. Various changes can be made to elements, or deletions can be made. In addition to the modifications described above, some further modifications are exemplified, but not exhaustive, and other modifications are possible. Two or more of these changes may be combined as appropriate, and may be combined with the changes described above.
In the treatment method according to the present disclosure, the tube 410 of the catheter 401 may be inserted from the third port 353 as shown in FIGS. 27 and 28. In this case, the insertion of the endoscope 500 from the main port 350, the aspiration of the gas in the closed space Cs, and the supply of the medicine to the closed space Cs are performed.
In the treatment method according to the present disclosure, as shown in FIG. 29, aspiration in step C may be performed using an aspiration catheter 601. In this case, the aspiration in step C and the delivery of the medicine in step D may be performed partially in parallel or completely simultaneously. Note that the overtube 301 may have a sub-lumen for aspiration.
In the treatment method according to the present disclosure, as shown in FIG. 30, when the catheter 401 is inserted into the main lumen 311 of the overtube 301, a balloon cover 430 may be located around at least the first balloon 431. Even if the first balloon 431 is made of a highly viscous material, it can be prevented from sticking to the inner peripheral surface of the main lumen 311 of the overtube 301 and being damaged. The outer peripheral surface of the balloon cover 430 is desirably coated with a lubricating coating such as hydrophilic lubrication.

### Third Embodiment

A third embodiment of the present disclosure will be described with reference to FIGS. 31 to 33. In the following description, the same reference numerals are given to the same configurations as those already described, and redundant descriptions will be omitted.

FIG. 31 is a schematic diagram of a device 701 used in the treatment method according to this embodiment. The treatment method according to this embodiment is not limited to using the device 701, but can be performed by using the device 701.

The device 701 is the same as the device 1 of the first embodiment except that it does not have a third balloon (auxiliary balloon) 40 and it has a pressure monitor 770.

FIG. 32 is a schematic diagram of device 701 in which pressure monitor 770 operates.

The pressure monitor 770 is provided on the outer peripheral surface of the tube 10 sandwiched between the first balloon 31 and the second balloon 32. The pressure monitor 770 has a membrane structure and deforms according to the pressure inside the closed space Cs. The membrane structure has an opening and a membrane attached to the opening. When the pressure in the closed space Cs is below a predetermined value, the shape of the membrane is maintained. When the pressure in the closed space Cs is greater than a predetermined value, the membrane deforms into a concave shape inside the tube 10. The membrane structure is made of the same material as the balloon, for example. The membrane structure communicates with the main lumen 11, and the membrane structure deformed inward according to the pressure in the closed space Cs is observed with the endoscope 500.

In step D, the operator observes the pressure monitor (membrane structure) 770 with the endoscope 500, and when it is determined that the internal pressure in the closed space Cs has reached a predetermined pressure, stops the medicine supply. The method of observing the pressure monitor (membrane structure) 770 with the endoscope 500 can more accurately determine the internal pressure within the closed space Cs, compared to the method of measuring the internal pressure in the closed space Cs using the pressure gauge 105A of the first port 51 on the proximal side.

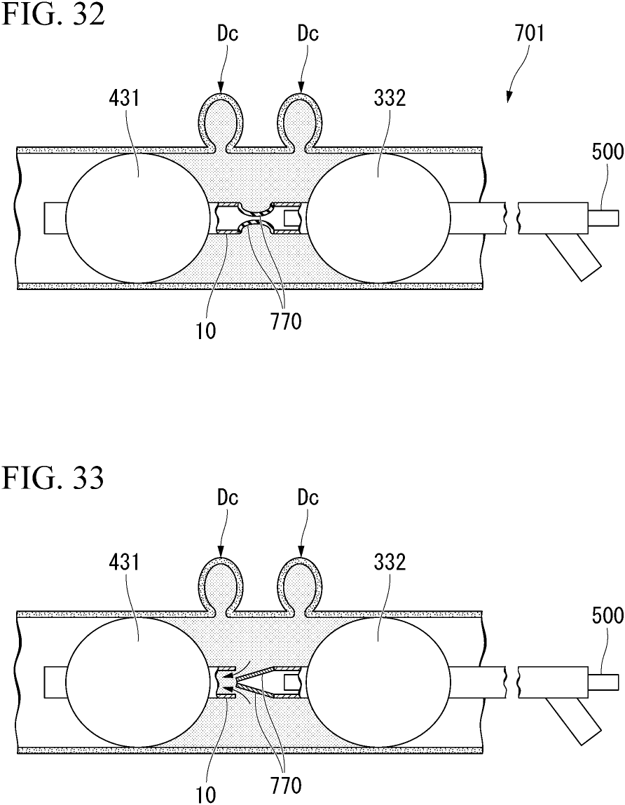

As described above, the third embodiment of the present disclosure has been described, but the technical scope of the present disclosure is not limited to the above-described embodiments, and the combination of components can be changed or each configuration can be changed without departing from the spirit of the present disclosure. Various changes can be made to elements, or deletions can be made. In addition to the modifications described above, some further modifications are exemplified, but not exhaustive, and other modifications are possible. Two or more of these changes may be combined as appropriate, and may be combined with the changes described above.
In the treatment method according to the present disclosure, the pressure monitor 770 may be a valve structure, as shown in FIG. 33. The valve structure communicates with the main lumen 11 and is released when the pressure in the closed space Cs reaches or exceeds a predetermined pressure. When the valve structure is opened, the medicine in the closed space Cs flows into the main lumen 11 and out through the distal opening. Treatment can be performed safely by preventing the internal pressure in the closed space Cs from exceeding a predetermined pressure. It is desirable that the operator observe the pressure monitor (valve structure) 770 with the endoscope 500 and stop the supply of the medicine according to the movement of the valve structure.

What is claimed is:

1. A treatment method, comprising:
inserting a first closure and a second closure into a tubular organ, wherein the tubular organ includes at least one affected site;
contacting an inner wall of the tubular organ with the first closure and the second closure to close the tubular organ with the at least one affected site located between the first closure and the second closure;
with the tubular organ closed by the first closure and the second closure, aspirating a gas located in the tubular organ between the first closure and the second closure; and
with the tubular organ closed by the first closure and the second closure, delivering a liquid medicine into the tubular organ between the first closure and the second closure.

2. The treatment method according to claim 1, wherein the tubular organ is a digestive tract, and
wherein the affected site is a diverticulum.

3. The treatment method according to claim 2, wherein the affected site is a colonic diverticulum, and
wherein the tubular organ includes a plurality of affected sites and the plurality of affected sites is located between the first closure and the second closure.

4. The treatment method according to claim 1, wherein contacting the inner wall of the tubular organ with the first closure and the second closure to close the tubular organ forms a closed space between the first closure and the second closure, and
wherein aspirating the gas results in a negative pressure in the closed space.

5. The treatment method according to claim 1, wherein delivering the liquid medicine into the tubular organ occurs after aspirating the gas.

6. The treatment method according to claim 1, wherein the first closure is a first balloon and the second closure is a second balloon,
wherein the first balloon and the second balloon are attached to a tube,
wherein the tube includes:
a first lumen communicating with the first balloon,
a second lumen communicating with the second balloon,
an opening located between the first balloon and the second balloon, and
a third lumen communicating with the opening, and
wherein the liquid medicine is delivered from the opening via the third lumen.

7. The treatment method according to claim 1, wherein the liquid medicine exerts a hemostatic effect.

8. The treatment method according to claim 1, wherein, after delivering the liquid medicine into the tubular organ, the treatment method further comprises removing at least a portion of the liquid medicine from the tubular organ.

9. The treatment method according to claim 1, wherein the affected site has a concave portion.

10. The treatment method according to claim 9, wherein aspirating the gas aspirates gas from inside the concave portion.

11. The treatment method according to claim 9, wherein delivering the liquid medicine into the tubular organ delivers the liquid medicine into the concave portion.

12. The treatment method according to claim 11, wherein delivering the liquid medicine into the tubular organ suppress bleeding in the affected site.

13. The treatment method according to claim 6, wherein the tube has a third balloon between the first closure and the second closure, and
wherein delivering the liquid medicine into the tubular organ occurs with the third balloon inflated.

14. A treatment method, comprising:
inserting a tube including a first closure and a second closure into a tubular organ including at least one diverticulum;
in a state in which the diverticulum is located between the first closure and the second closure, contacting an inner wall of the tubular organ with the first closure and the second closure to form a closed space between the first closure and the second closure;
aspirating a gas in the diverticulum and the closed space to place the closed space under a negative pressure; and
delivering a liquid medicine into the closed space to suppress bleeding in the diverticulum.

15. A treatment method, comprising:
inserting a catheter including a first closure and an overtube including a second closure into a tubular organ including at least one affected site;
contacting an inner wall of the tubular organ with the first closure and the second closure to close the tubular organ with the at least one affected site located between the first closure and the second closure;
with the tubular organ closed by the first closure and the second closure, aspirating a gas located in the tubular organ between the first closure and the second closure; and
with the tubular organ closed by the first closure and the second closure, delivering a liquid medicine into the tubular organ between the first closure and the second closure.

16. The treatment method according to claim 15, further comprising retracting and advancing the catheter relative to the overtube to adjust a position of the first closure relative to the second closure.

17. The treatment method according to claim 15, further comprising pulling the catheter proximally relative to the overtube to move the first closure proximally relative to the second closure.

18. The treatment method according to claim 6, wherein contacting the inner wall of the tubular organ with the first closure and the second closure to close the tubular organ forms a closed space between the first closure and the second closure,
wherein the tube includes a membrane structure deformable according to a pressure inside the closed space, and
wherein the treatment method further comprises:
observing the membrane structure with an endoscope, and
stopping delivering the liquid medicine when the membrane structure is deformed.

19. The treatment method according to claim 6, wherein contacting the inner wall of the tubular organ with the first closure and the second closure to close the tubular organ forms a closed space between the first closure and the second closure,
wherein the tube includes a valve that releases when a pressure in the closed space exceeds a predetermined pressure inside the closed space, and
wherein the treatment method further comprises:
observing the valve with an endoscope, and
stopping delivering the liquid medicine when the valve is released.
